(19) United States
(12) Patent Application Publication
(10) Pub. No.: US 2003/0232014 A1
(43) Pub. Date: Dec. 18, 2003

(54) Phosphorylated proteins and uses related thereto

(75) Inventors: Daniel J. Burke, Charlottesville, VA (US); Mark M. Ross, Charlottesville, VA (US); P. Todd Stukenberg, Charlottesville, VA (US); Forest M. White, Charlottesville, VA (US)

Correspondence Address: Matthew P. Vincent, Ropes & Gray, One International Place, Boston, MA, 02110-2624, US

(73) Assignees: MDS Proteomics Inc., Toronto (CA); University of Virginia, Charlottesville, VA

(21) Appl. No.: 10/378,173

(22) Filed: Mar. 3, 2003

Related U.S. Application Data
(60) Provisional application No. 60/360,787, filed on Mar. 1, 2002.

(51) Int. Cl. A61K 51/00; C07K 16/18; C07K 14/00

(52) U.S. Cl. 424/1.69; 530/400; 530/388.1; 705/2

(57) ABSTRACT

Methods and systems of applying mass spectrometry to the analysis of peptides and amino acids, especially in the proteome setting. More particularly, the invention relates to a mass spectrometry-based method for detection of amino acid modifications, such as phosphorylation.

### REFERENCE OF RELATED APPLICATIONS

This application claims priority to U.S. Provisional Application No. 60/360,787, filed on Mar. 1, 2002, the entire content of which is incorporated herein by reference.

### BACKGROUND OF THE INVENTION

With the availability of a burgeoning sequence database, genomic applications demand faster and more efficient methods for the global screening of protein expression in cells. However, the complexity of the cellular proteome expands substantially if protein post-translational modifications are also taken into account.

Dynamic post-translational modification of proteins is important for maintaining and regulating protein structure and function. Among the several hundred different types of post-translational modifications characterized to date, protein phosphorylation plays a prominent role. Enzyme-catalyzed phosphorylation and dephosphorylation of proteins is a key regulatory event in the living cell. Complex biological processes such as cell cycle, cell growth, cell differentiation, and metabolism are orchestrated and tightly controlled by reversible phosphorylation events that modulate protein activity, stability, interaction and localization. Perturbations in phosphorylation states of proteins, e.g. by mutations that generate constitutively active or inactive protein kinases and phosphatases, play a prominent role in oncogenesis. Comprehensive analysis and identification of phosphoproteins combined with exact localization of phosphorylation sites in those proteins (‘phosphoproteomics’) is a prerequisite for understanding complex biological systems and the molecular features leading to disease.

Protein phosphorylation represents one of the most prevalent mechanisms for covalent modification. It is estimated that one third of all proteins present in a mammalian cell are phosphorylated and that kinases, enzymes responsible for that phosphorylation, constitute about 1-3% of the expressed genome. Organisms use reversible phosphorylation of proteins to control many cellular processes including signal transduction, gene expression, the cell cycle, cytoskeletal regulation and apoptosis. A phosphate group can modify serine, threonine, tyrosine, histidine, arginine, lysine, cysteine, glutamic acid and aspartic acid residues. However, the phosphorylation of hydroxyl groups at serine (90%), threonine (10%), or tyrosine (0.05%) residues are the most prevalent, and are involved among other processes in metabolism, cell division, cell growth, and cell differentiation. Because of the central role of phosphorylation in the regulation of life, much effort has been focused on the development of methods for characterizing protein phosphorylation.

### REFERENCE TO THE DRAWINGS

FIG. 1. Five nonphosphorylated proteins; glyceraldehyde 3-phosphate dehydrogenase, bovine serum albumin, carbonic anhydrase, ubiquitin, and β-lactoglobulin (Sigma Chemical Co., St. Louis, Mo.) (100 nmol each) in 1.1 ml of 100 mM ammoniun bicarbonate (pH 8) were digested with trypsin (20 μg) (Promega, Madison, Wis.) for 24 h at 37° C. The reaction was quenched with 65 μl of glacial acetic acid, and the mixture was then diluted to final volume of 50 ml with 0.1% acetic acid. To this solution was added 500 pmol of HPLC purified phosphopeptide, DRVPYIHPF (SEQ ID NO: 1, Novabiochem, San Diego, Calif.), in 0.1% acetic acid (2 μL of a 250 pmol/μL stock solution). An aliquot of the standard mixture (100 μl) was lyophilized and redissolved in 100 μl of 2 N methanolic HCl. This latter solution was prepared by dropwise addition of 160 μl of acetyl chloride with stirring to 1 ml of methanol. Esterification was allowed to proceed for 2 h at room temperature. Solvent was removed by lyophylization and the resulting sample was redissolved in 100 μl of solution containing equal volumes of methanol, water and acetonitrile. Phosphate methyl esters are not observed under these conditions. Mass spectra recorded by a combination of immobilized metal affinity chromatography (IMAC) and nano-flow HPLC microelectrospray ionization mass spectrometry on the phosphopeptide, DRVpYIHPF (SEQ ID NO: 1), present at the level of 10 fmol/μl in a mixture containing tryptic peptides from 5 proteins at the level of 2 pmol/μl. Aliquots corresponding to 0.5 μl of the above solutions (tryptic peptides from 1 pmol of each protein plus 5 fmol of phosphopeptide, DRVpYIHPF, SEQ ID NO: 1) were analyzed by mass spectrometry. (A) Selected ion chromatogram, SIC, or plot of the ion current vs scan number for m/z 564.5 corresponding to the (M+2H)⁺⁺ of the phosphopeptide, DRVpYIHPF (SEQ ID NO: 1). (B) MS/MS spectrum characteristic of the sequence, DRVpYIHPF (SEQ ID NO: 1), recorded on ions of m/z 564.5 in scans 610-616. (C) Electrospray ionization mass spectrum recorded during this same time interval. Abundant ions from tryptic peptides non-specifically bound to the IMAC column obscure the signal at m/z 564.5 for DRVpYIHPF (SEQ ID NO: 1). (D) SIC for m/z 578.5 corresponding to the (M+2H)⁺⁺ ion for the dimethyl ester of DRVpYIPF (SEQ ID NO: 1). (E) MS/MS spectrum characteristic of the sequence, DRVpYIPF (SEQ ID NO: 1), recorded in on ions of m/z 578.5 in scans 151-163. (F) Electrospray ionization mass spectrum recorded in scan 154 showing the parent ion, m/z 578.5 for the phosphopeptide dimethyl ester and the absence of signals for tryptic peptides non specifically bound to the IMAC column.

### DETAILED DESCRIPTION OF THE INVENTION

I. Overview

The present invention relates to the identification of phosphorylated proteins from eukaryotic cells. As described in further detail below, more than a 1,000 phosphopeptides were identified during the analysis of a whole cell lysate from S. cerevisiae. Phosphopeptide sequences, including 383 sites of phosphorylation derived from 216 peptides were determined. Of these 60 were singly phosphorylated, 145 doubly phosphorylated, and 11 triply phosphorylated. In addition to the identified sequences, the present invention specifically contemplates that the same or similar sequences exist in corresponding mammalian proteins, especially human proteins, and are included in the term “phosphopeptide sequence” as used herein.

The discovery of these phosphorylated proteins provides several advantages, including compositions of peptides and polypeptides which include one or more of the subject phosphopeptide sequences. The phosphopeptide sequence can be provided as a peptide, e.g., having 4 or more residues, and can also be present as a monomeric sequence in a larger polypeptide, or can be present in multiple copies having the same or different amino acid sequences. Moreover, the phosphopeptide sequence is a modular component, and can be added at various positions to a chimeric protein with no more than routine experimentation.

In certain embodiments, the subject peptides and polypeptides can be used as substrates for kinases (when unphosphorylated) or phosphatases (when phosphorylated), or binding moieties for SH2 domains (when phosphorylated), and can be used in assays for identifying agents which potentiate or inhibit the activity of the kinase, phosphatase or SH2-containing proteins. In other embodiments, the subject peptides and polypeptides can be used a inhibitors of kinase, phosphatase or SH2-containing proteins.

Another aspect of the invention provides a peptide or peptidomimetic, e.g., wherein one or more backbone bonds is replaced or one or more sidechains of a naturally occurring amino acid are replaced with sterically and/or electronically similar functional groups.

An certain embodiments, the peptide or peptidomimetic is formulated in a pharmaceutically acceptable excipient.

Another aspect of the invention relates to a nucleic acid encoding a polypeptide which includes one or more phosphopeptide sequence.

Yet another aspect of the invention relates to a pharmaceutical preparation comprising a therapeutically effective amount of a phosphopeptide or peptidomimetic, formulated in the pharmaceutical preparation for delivery into cells of an animal.

II. Definitions

For convenience, certain terms employed in the specification, examples, and appended claims are collected here.

As used herein, the term “gene” or “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides.

The terms “protein”, “polypeptide” and “peptide” are used interchangeably herein when referring to a gene product, e.g., as may be encoded by a coding sequence.

As used herein, the term “transfection” means the introduction of a nucleic acid, e.g., an expression vector, into a recipient cell by nucleic acid-mediated gene transfer.

“Transcriptional regulatory sequence” is a generic term used throughout the specification to refer to DNA sequences, such as initiation signals, enhancers, and promoters, which induce or control transcription of protein coding sequences with which they are operably linked.

Operably linked is intended to mean that the nucleotide sequence is linked to a regulatory sequence in a manner which allows expression of the nucleotide sequence. Regulatory sequences are art-recognized and are selected to direct expression of the subject peptide. Accordingly, the term transcriptional regulatory sequence includes promoters, enhancers and other expression control elements. Such regulatory sequences are described in Goeddel; Gene Expression Technology: Method in Enzymology 185, Academic Press, San Diego, Calif. (1990).

The term “gene construct” refers to a vector, plasmid, viral genome or the like which includes a coding sequence, can transfect cells, preferably mammalian cells, and can cause expression of a peptide or polypeptide including a phosphopeptide sequence in the cells transfected with the construct.

The term “interact” as used herein is meant to include detectable interactions between molecules, such as can be detected using, for example, a yeast two hybrid assay or by immunoprecipitation. The term interact is also meant to include “binding” interactions between molecules. Interactions may be, for example, protein-protein, protein-nucleic acid, protein-small molecule or small molecule-nucleic acid in nature. Preferred binding affinities have a K_{d} of 10⁻⁶ M or less, preferably 10⁻⁸ or less, 10⁻⁹ or less, 10⁻¹⁰ or less, 10⁻¹¹ or less, or most preferably 10⁻¹² or less.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of“plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The terms “chimeric”, “fusion” and “composite” are used to denote a protein, peptide domain or nucleotide sequence or molecule containing at least two component portions which are mutually heterologous in the sense that they are not, otherwise, found directly (covalently) linked in nature. More specifically, the component portions are not found in the same continuous polypeptide or gene in nature, at least not in the same order or orientation or with the same spacing present in the chimeric protein or composite domain. Such materials contain components derived from at least two different proteins or genes or from at least two non-adjacent portions of the same protein or gene. Composite proteins, and DNA sequences which encode them, are recombinant in the sense that they contain at least two constituent portions which are not otherwise found directly linked (covalently) together in nature.

The term “amino acid residue” is known in the art. In general the abbreviations used herein for designating the amino acids and the protective groups are based on recommendations of the IUPAC-IUB Commission on Biochemical Nomenclature (see Biochemistry (1972)11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisoiners of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers. D- and L-α-Amino acids are represented by the following Fischer projections and wedge-and-dash drawings. In the majority of cases, D- and L-amino acids have R- and S-absolute configurations, respectively.

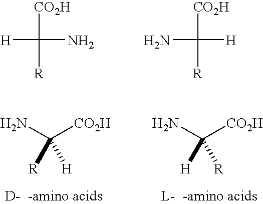

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g. the ability to bind to a kinase, phosphatase and/or SH2 domain), wherein one or more simple variations of substituents are made which do not adversely affect the efficacy of the compound in, for example, acting as a substrate or inhibitor of a kinase, phosphatase and/or SH2 domain. In general, the compounds of the present invention may be prepared by the methods illustrated in the general reaction schemes as, for example, described below, or by modifications thereof, using readily available starting materials, reagents and conventional synthesis procedures. Thus, the contemplated equivalents include peptidomimetic or non-peptide small molecules. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

As used herein, the term “pharmaceutically acceptable” refers to a carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitonieal, with intravenous being preferred.

As used herein, the term “prophylactic or therapeutic” treatment refers to administration to the host of the medical condition. If it is administered prior to exposure to the condition, the treatment is prophylactic (i.e., it protects the host against infection), whereas if administered after infection or initiation of the disease, the treatment is therapeutic (i.e., it combats the existing infection or cancer).

III. Description of Certain Preferred Embodiments

A. Chimeric Phosphopeptide Peptides and Peptidomimetic

In addition to the use of the subject native phosphopeptides and full length wild-type proteins in which they occur, the invention also provides chimeric proteins which include one or more phosphopeptide fused to one or more additional protein domains. In one embodiment, the chimeric protein includes one phosphopeptide sequence. In other embodiments, the chimeric activator comprises two or more phosphopeptide sequences, three or more, five or more, or ten or more phosphopeptide sequences that are covalently linked. When referring to a polypeptide comprising a phosphopeptide sequence, it is meant that the polypeptide comprises the amino acid sequence of a phosphopeptide covalently linked to other amino acids or peptides to form one polypeptide. The order of the phosphopeptide(s) relative to each other and relative to the other domains of the fusion protein can be as desired.

Techniques for making the subject fusion proteins are adapted from well-known procedures. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments.

Amplification products can subsequently be annealed to generate a chimeric gene sequence (see, for example, Current Protocols in Molecular Biology, Eds. Ausubel et al. John Wiley & Sons: 1992).

In certain embodiments, polyanionic or polycatonic binding agents such as oligonucleotides, heparin, lentinan and similar polysaccharide chains, polyamino peptides such as polyaspartate, polyglutamate, polylysine and polyarginine, or other binding agents which maintain a number of either negative or positive charges over their structure at physiological pH's, can be used to specifically bind the the subject phosphopeptide or peptidomimetics. In certain preferred embodiments, a polyanionic component is used, such as heparin, pentosan polysulfate, polyaspartate, polyglutamate, chondroitin sulfate, heparan sulfate, citrate, nephrocalcin, or osteopontin, to name but a few.

(i) Additional Domains and Linkers

Additional domains may be included in the subject fusion proteins of this invention. For example, the fusion proteins may include domains that facilitate their purification, e.g. “histidine tags” or a glutathione-S-transferase domain. They may include “epitope tags” encoding peptides recognized by known monoclonal antibodies for the detection of proteins within cells or the capture of proteins by antibodies in vitro.

It may be necessary in some instances to introduce an unstructured polypeptide linker region between a phosphopeptide and other portions of the chimeric protein. The linker can facilitate enhanced flexibility of the fusion protein. The linker can also reduce steric hindrance between any two fragments of the fusion protein. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the lcI and LexA proteins. Alternatively, the linker can be of synthetic origin. For instance, the sequence (Gly₄Ser)₃, SEQ ID NO: 2, can be used as a synthetic unstructured linker. Linkers of this type are described in Huston et al. (1988) PNAS 85:4879; and U.S. Pat. No. 5,091,513.

In some embodiments it is preferable that the design of a linker involve an arrangement of domains which requires the linker to span a relatively short distance, preferably less than about 10 Angstrom. However, in certain embodiments, depending, e.g., upon the selected domains and the configuration, the linker may span a distance of up to about 50 Angstrom.

Within the linker, the amino acid sequence may be varied based oil the preferred characteristics of the linker as determined empirically or as revealed by modeling. For instance, in addition to a desired length, modeling studies may show that side groups of certain amino acids may interfere with the biological activity of the fusion protein. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. For example, a linker may contain an amino acid sequence which can be recognized by a protease so that the activity of the chimeric protein could be regulated by cleavage. In some cases, particularly when it is necessary to span a longer distance between subunits or when the domains must be held in a particular configuration, the linker may optionally contain an additional folded domain.

(ii) Toxins and Imaging Agents

In certain embodiments, the subject phosphopeptides and peptidomimetics can be covalently or non-covalently coupled to a cytotoxin or other cell proliferation inhibiting compound, in order to localize delivery of that agent to a particular cell or tissue type. For instance, the agent can be selected from the group consisting of alkylating agents, enzyme inhibitors, proliferation inhibitors, lytic agents, DNA or RNA synthesis inhibitors, membrane permeability modifiers, DNA intercalators, metabolites, dichloroethylsulfide derivatives, protein production inhibitors, ribosome inhibitors, inducers of apoptosis, and neurotoxins.

Chemotherapeutics useful as active moieties when conjugated to a modified phosphopeptides or peptidomimetics will typically include small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents useful in the present invention are listed, for example, in Goodman et al., “The Pharmacological Basis of Therapeutics,” Sixth Edition, A. G. Gilnan et al, eds./Macmillan Publishing Co. New York, 1980. These include taxanes, such as paclitaxel (Taxol®) and docetaxel (Taxotere®); nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as inethotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to these skilled in the art and include puromycin, cycloheximide, and ribonuclease.

Prodrugs forms of the chemotherapeutic moiety are especially useful in the present invention to generate an inactive precursor.

Most of the chemotherapeutic agents currently in use in treating cancer possess functional groups that are amenable to chemical crosslinking directly with an amine or carboxyl group of a phosphopeptide. For example, free amino groups are available on methotrexate, doxorubicin, daunorubicin, cytosinarabinoside, bleomycin, gemcitabine, fludarabine, and cladribine while free carboxylic acid groups are available on methotrexate, melphalan, and chlorambucil. These functional groups, that is free amino and carboxylic acids, are targets for a variety of homobifunctional and heterobifunctional chemical crosslinking agents which can crosslink these drugs directly to a free amino group of a phosphopeptide.

Peptide and polypeptide toxins are also useful as active moieties, and the present invention specifically contemplates embodiments wherein the phosphopeptide moiety is coupled to a toxin. In certain preferred embodiments, the phosphopeptide and toxin are both polypeptides and are provided in the form of a fusion protein. Toxins are generally complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

The invention further contemplates embodiments the phosphopeptide is coupled to a polymer or a functionalized polymer (e.g., a polymer conjugated to another molecule). Preferred examples include water soluble polymers, such as, polyglutamic acid or polyaspartic acid, conjugated to a drug such as a chemotherapeutic or antiangiogenic agent, including, for example, paclitaxel or docetaxel.

In addition, there are other active agents which can be used to create a modified phosphopeptide. For example, modified phosphopeptide can be generated to include active enzyme. The modified phosphopeptide specifically localizes the activity to particular cells. An inactive prodrug which can be converted by the enzyme into an active drug is administered to the patient. The prodrug is only converted to an active drug by the enzyme which is localized to the cell. An example of an enzyme/prodrug pair includes alkaline phosphatase/etoposidephosphate. In such a case, the alkaline phosphatase is conjugated to a phosphopeptide. The modified phosphopeptide is administered and localizes to targeted cancer cells. Upon contact with etoposidephosphate (the prodrug), the etoposidephosphate is converted to etoposide, a chemotherapeutic drug which is taken up by the cancer cell.

In certain preferred embodiments, particularly where the cytotoxic moiety is chemically cross-linked to the peptide moiety, the linkage is hydrolyzable from the peptide, e.g., such as may be provided by use of an amide or ester group in the linking moiety.

In certain embodiments, the subject peptides and peptidomimetics can be coupled with an agent useful in imaging. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores. In many embodiments, such secondary functionality will be relatively large, e.g., at least 25 amu in size, and in many instances can be at least 50, 100 or 250 amu in size.

In certain preferred embodiments, the secondary functionality is a chelate moiety for chelating a metal, e.g., a chelator for a radiometal or paramagnetic ion. In preferred embodiments, it is a chelator for a radionuclide useful for radiotherapy or imaging procedures. Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ³²P, ³³P, ⁴³K, ⁴⁷Sc, ⁵²Fe, ⁵⁷Co, ⁶⁴Cu, ⁶⁷Ga ⁶⁷CU ⁶⁸Ga ⁷¹Ge ⁷⁵Br ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁵I, ¹²⁷Cs, ¹²⁸Ba, ¹²⁹Cs, ¹³¹I, ¹³¹Cs, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹¹⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chelator will coordinate a metal are described, for example, by Gansow et al., U.S. Pat. Nos. 4,831,175, 4,454,106 and 4,472,509

^{99m}Tc is a particularly attractive radioisotope for therapeutic and diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the modified phosphopeptide includes a chelating agent for technium.

In still other embodiments, the secondary functionality can be a radiosensitizing agent, e.g., a moiety that increase the sensitivity of cells to radiation. Examples of radiosensitizing agents include nitroimidazoles, metronidazole and misonidazole (see: DeVita, V. T. Jr. in Harrison's Principles of Internal Medicine, p.68, McGraw-Hill Book Co., N.Y. 1983, which is incorporated herein by reference). The modified phosphopeptide that comprises a radiosensitizing agent as the active moiety is administered and localizes at the metastasized cell. Upon exposure of the individual to radiation, the radiosensitizing agent is “excited” and causes the death of the cell.

There are a wide range of moieties which can serve as chelators and which can be derivatized to the phosphopeptide. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to a phosphopeptide. Such groups include, e.g., benzylisothiocyanate, by which the DOTA, DTPA or EDTA can be coupled to, e.g., an amine group of the phosphopeptide.

In one embodiment, the chelate moiety is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85:4024-29; and Weber et al. (1990) Bioconiugate Chem. 1:431-37; and in the references cited therein.

The Jacobsen et al. PCT application WO 98/12156 provides methods and compositions, i.e. synthetic libraries of binding moities, for identifying compounds which bind to a metal atom. The approach described in that publication can be used to identify binding moieties which can subsequently be added to phosphopeptides to derive the modified phosphopeptides of the present invention.

A problem frequently encountered with the use of conjugated proteins in radiotherapeutic and radiodiagnostic applications is a potentially dangerous accumulation of the radiolabeled moiety fragments in the kidney. When the conjugate is formed using a acid-or base-labile linker, cleavage of the radioactive chelate from the protein can advantageously occur. If the chelate is of relatively low molecular weight, as most of the subject modified phosphopeptides are expected to be, it is not retained in the kidney and is excreted in the urine, thereby reducing the exposure of the kidney to radioactivity. However, in certain instances, it may be advantageous to utilize acid-or base-labile linkers in the subject ligands for the same reasons they have been used in labeled proteins.

Accordingly, certain of the subject modified phosphopeptides can be synthesized, by standard methods known in the art, to provide reactive functional groups which can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively.

Alternatively, base-cleavable linkers, which have been used for the enhanced clearance of the radiolabel from the kidneys, can be used. See, for example, Weber et al. 1990 Bioconjug. Chem. 1:431. The coupling of a bifunctional chelate to a phosphopeptide via a hydrazide linkage can incorporate base-sensitive ester moieties in a linker spacer arm. Such an ester-containing linker unit is exemplified by ethylene glycolbis(succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NHS ester is reacted with a limiting amount of hydrazine. The resulting hyrazide is used for coupling to the phosphopeptide, forming an ligand-BFC linkage containing two alkyl ester functions. Such a conjugate is stable at physiological pH, but readily cleaved at basic pH.

Phosphopeptide labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the peptide or peptidomimetic is coupled to a Boron addend, such as a carborane. For example, carboranes can be prepared with carboxyl functions on pendant side chains, as is well known in the art. Attachment of such carboranes to an amine functionality, e.g., as may be provided on the phosphopeptide, can be achieved by activation of the carboxyl groups of the carboranes and condensation with the amine group to produce the conjugate. Such modified phosphopeptides can be used for neutron capture therapy.

The present invention also contemplates the modification of the subject peptides with dyes, for example, useful in photodynamic therapy, and used in conjunction with appropriate non-ionizing radiation. The use of light and porphyrins in methods of the present invention is also contemplated and their use in cancer therapy has been reviewed. van den Bergh, Chemistry in Britain, 22: 430-437 (1986), which is incorporated herein in its entirety by reference.

(iii) Peptide Vaccines

In still other embodiments, the subject phosphopeptides can be used to generate vaccines, e.g., against tumor or other cells in which the phosphopeptide sequences selectively occur. For instance, the subject invention concerns vaccines comprising an immunogenic formulation of one or more phosphopeptides or proteins which include a phosphopeptide sequence.

It will be readily appreciated that the phosphopeptide of this invention can be incorporated into vaccines capable of inducing protective immunity against certain cells in which the phosphopeptide moiety occurs as part of the etiology of disease. Phosphopeptide-derived vaccines may be synthesized or prepared recombinantly or otherwise biologically, to comprise one or more phosphopeptide amino acid sequences' corresponding to one or more epitopes of the phosphopeptide sequence either in monomeric or multimeric form. Those proteins and/or polypeptides may then be incorporated into vaccines capable of inducing protective immunity. Techniques for enhancing the antigenicity of such polypeptides include incorporation into a multimeric structure, binding to a highly immunogenic protein carrier, for example, keyhole limpet hemocyanin (KLH), or diptheria toxoid, and administration in combination with adjuvants or any other enhancers of immune response.

B. Phosphopeptide Peptidomimetics

In other embodiments, the subject invention contemplates peptidomimetics of the phosphopeptide sequences. Peptidomimetics are compounds based on, or derived from, peptides and proteins. The phosphopeptide peptidomimetics of the present invention typically can be obtained by structural modification of a known phosphopeptide sequence using unnatural amino acids, conformational restraints, isosteric replacement, and the like. The subject peptidomimetics constitute the continum of structural space between peptides and non-peptide synthetic structures; phosphopeptide peptidomimetics may be useful, therefore, in delineating pharmacophores and in helping to translate peptides into nonpeptide compounds with the activity of the parent phosphopeptide.

Moreover, as is apparent from the present disclosure, mimetopes of the subject phosphopeptide can be provided. Such peptidomiimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides. Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1: 1231), β-aminoalcohols (Gordon et al. (1985) Biochem Biophys Res Commun126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides. Chemistry and Biology, G. R. Marshall ed., ESCOM Publisher: Leiden, Netherlands, 1988)

In addition to a variety of sidechain replacements which can be carried out to generate the subject phosphopeptide peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

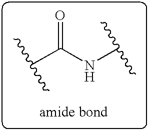

Examples of Surrogates

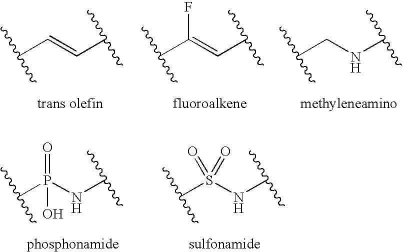

Additionally, peptidomimietics based on more substantial modifications of the backbone of the E2 peptide can be used. Peptidomimetics which fall in this category include (i) retro-inverso analogs, and (ii) N-alkyl glycine analogs (so-called peptoids).

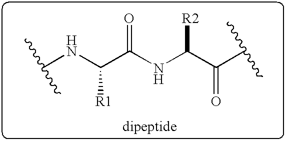

Examples of Analogs

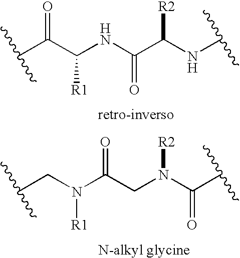

Furthermore, the methods of combinatorial chemistry are being brought to bear, e,g, by G. L. Verdine at Harvard University, on the development of new peptidomimetics. For example, one embodiment of a so-called “peptide morphing” strategy focuses on the random generation of a library of peptide analogs that comprise a wide range of peptide bond substitutes.

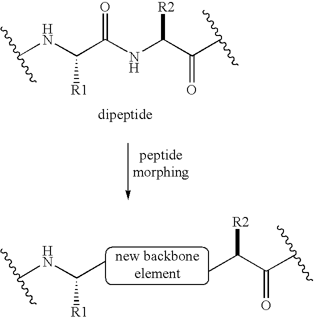

In an exemplary embodiment, the peptidomimetic can be derived as a retro-inverso analog of the peptide

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching The final product, or intermediates thereof, can be purified by HPLC.

In another illustrative embodiment, the peptidomimetic can be derived as a retro-enatio analog of-a particular phosphopeptide sequence. Retro-enantio analogs such as this can be synthesized commercially available D-amino acids (or analogs thereof) and standard solid- or solution-phase peptide-synthesis techniques.

In still another illustrative embodiment, trans-olefin derivatives can be made for any of the subject polypeptides. A trans-olefin analog of phosphopeptide can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible couple the pseudodipeptides synthesized by the above method to other pseudodipeptides, to make peptide analogs with several olefinic functionalities in place of amide functionalities. For example, pseudodipeptides corresponding to certain di-peptide sequences could be made and then coupled together by standard techniques to yield an analog of the phosphopeptide which has alternating olefinic bonds between residues.

Still another class of peptidomimetic derivatives include phosphonate derivatives. The synthesis of such phosphonate derivatives can be adapted from known synthesis schemes. See, for example, Loots et al. in Peptides. Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the the subject phosphopeptide peptidomimetics. To illustrate, the phosphopeptide peptidomimetic may incorporate the 1-azabicyclo[4.3.0]nonane surrogate ( see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

In certain embodiments, non-hydrolyzable analogs of the phosphorylated amino acid residue can be used. For instance, the phosphate group on a phosphoserine, phosphothreonine or phosphotyrosine can be replaced with a moiety selected from the group consisting of:

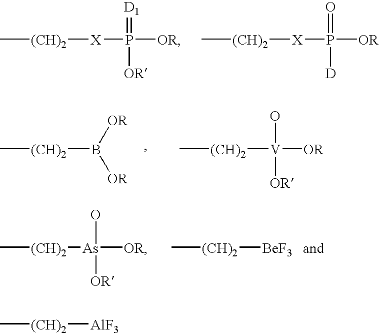

wherein m is zero or an integer in the range of 1 to 6; X is absent (a bond) or represents O, S, or N; D₁ represents O, or S; D₂ represents N₃, SH₂, NH₂, or NO₂; and R and R′ independently for each occurrence represent hydrogen, a lower alkyl, or a pharmaceutically acceptable salt, or R and R′ taken together with the O—P—O, O—B—O, O—V—O or O—As—O atoms to which they are attached complete a heterocyclic ring having from 5 to 8 atoms in the ring structure.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to a cognate binding partner of the phosphopeptide e.g., and competitively inhibiting the interaction between the phosphopeptide and cognate binding partner. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. the predicted mimetope structures can then be produced by, for example, chemical synthesis, recombinant DNA technology, or by isolating a mimetope from a natural source (e.g., plants, animals, bacteria and fungi).

C. Generating Variants of Phosphopeptide Sequences

In addition to the naturally occurring phosphopeptide sequences (yeast, human or otherwise), the peptide compositions of the present invention include other peptidomimetics, non-peptide small molecules, genes and recombinant polypeptides that may be generated using any of a variety of combinatorial techniques for generating combinatorial libraries of small organic/peptide libraries. See, for example, Blondelle et al. (1995) Trends Anal. Chem. 14:83; the Affymax U.S. Pat. Nos. 5,359,115 and 5,362,899; the Ellman U.S. Pat. 5,288,514; the Still et al. PCT publication WO 94/08051; Chen et al. (1994) JACS 116:2661; Kerr et al. (1993) JACS 115:252; PCT publications WO92/10092, WO93/09668 and WO91/07087; and the Lerner et al. PCT publication WO93/20242).

In an exemplary embodiment, a combinatorial peptide library of potential phosphopeptide sequences can be produced by way of a degenerate library of genes encoding a library of polypeptides which each include at least a portion of potential phosphopeptide sequences. For instance, a mixture of synthetic oligonucleotides can be enzymatically ligated into gene sequences such that the degenerate set of potential phosphopeptide coding sequences are expressible as individual polypeptides, or alternatively, as a set of larger fusion proteins (e.g. for phage display) containing the set of phosphopeptide sequences therein.

There are many ways by which the gene library of potential phosphopeptide sequences can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential phosphopeptide sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations. Such techniques will be generally adaptable for rapid screening of the gene libraries generated by the combinatorial mutagenesis of phosphopeptide sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques.

In an illustrative embodiment of a screening assay, the phosphopeptide gene library can be expressed as a fusion protein on the surface of a viral particle. For instance, in the filamentous phage system, foreign peptide sequences can be expressed on the surface of infectious phage, thereby conferring two significant benefits. First, since these phage can be applied to immobilized kinases, antibodies or other binding agents at very high concentrations, a large number of phage can be screened at one time. Second, since each infectious phage displays the combinatorial gene product on its surface, if a particular phage is recovered by affinity purification in low yield, the phage can be amplified by another round of infection. The group of almost identical E.coli filamentous phages M13, fd, and fl are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461).

Merely to illustrate, the recombinant phage antibody system (RPAS, Pharmacia Catalog number 27-9400-01) can be easily modified for use in expressing and screening phosphopeptide motif libraries of the present invention. For instance, the pCANTAB 5 phagemid of the RPAS kit contains the gene which encodes the phage gIII coat protein. A library of potential phosphopeptide sequences can be cloned into the phagemid adjacent to the gill signal sequence such that it will be expressed as a gIII fusion protein. After ligation, the phagemid is used to transform competent E. coli TG1 cells. Transformed cells are subsequently infected with M13KO7 helper phage to rescue the phagemid and its candidate phosphopeptide gene insert. The resulting recombinant phage contain phagemid DNA encoding a specific candidate phosphopeptide, and display one or more copies of the corresponding fusion coat protein. In one embodiment, the phage-displayed candidate proteins which are capable of binding to immobilized kinase(s) are selected or enriched by panning. In other embodiments, the phage products can be treated with one or more kinases, and the phosphorylated peptide sequences can be isolated by the ability of the phage particle to be affinity purified using SH2 domains or anti-phosphopeptide antibodies. The isolated phage retain the ability to infect E. coli. Thus, successive rounds of reinfection of E. coli , and panning will greatly enrich for phosphopeptide sequences which can then be screened for sequences which retain the ability to be a kinase substrate or an SH2 domain binding motif.

D. Nucleic Acid Compositions

In another aspect of the invention, the coding sequences for the subject peptides and polypeptides described herein are provided in expression vectors. For instance, expression vectors are contemplated which include a nucleotide sequence encoding a polypeptide containing at least one phosphopeptide sequence, which coding sequence is operably linked to at least one transcriptional regulatory sequence. Regulatory sequences for directing expression of the instant fusion proteins are art-recognized and are selected by a number of well understood criteria. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the fusion proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

As will be apparent, the subject gene constructs can be used to cause expression of the subject fusion proteins in cells propagated in culture, e.g. to produce proteins or polypeptides, including fusion proteins, for purification.

This invention also pertains to a host cell transfected with a recombinant gene in order to express one of the subject polypeptides. The host cell may be any prokaryotic or eukaryotic cell. For example, a fusion proteins of the present invention may be expressed in bacterial cells such as E. coli , insect cells (baculovirus), yeast, or mammalian cells. Other suitable host cells are known to those skilled in the art.

Accordingly, the present invention further pertains to methods of producing the subject fusion proteins. For example, a host cell transfected with an expression vector encoding a protein of interest can be cultured under appropriate conditions to allow expression of the protein to occur. The protein may be secreted, by inclusion of a secretion signal sequence, and isolated from a mixture of cells and medium containing the protein. Alternatively, the protein may be retained cytoplasmically and the cells harvested, lysed and the protein isolated. A cell culture includes host cells, media and other byproducts. Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

Thus, a coding sequence for a fusion protein of the present invention can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures.

Expression vehicles for production of a recombinant protein include plasmids and other vectors. For instance, suitable vectors for the expression of the instant fusion proteins include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eulkaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant fusion proteins by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the beta-gal containing pBlueBac III).

In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection 5 spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus straini Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner- et al., supra; and Graham et al., in Methods in Molecular Biology, E. J. Murray, Ed. (Humana, Clifton, N.J., 1991) vol. 7. pp. 109-127). Expression of the inserted chimeric (gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Ophthalmol Vis Sci 35:2662-2666)

In addition to viral transfer methods, such as those illustrated above, non-viral methods can also be employed to cause expression of a protein in the tissue of an animal. Most noniviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding a phosphopeptide-containing polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/203 16, WO92/19749, and WO92/06180). For example, any of the subject gene constructs can be used to transfect specific cells in vivo using a soluble polynucleotide carrier comprising an antibody conjugated to a polycation, e.g. poly-lysine (see U.S. Pat. No. 5,166,320). It will also be appreciated that effective delivery of the subject nucleic acid constructs via -mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art.

For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

E. Exemplary Uses

The compositions and methods of the present invention are useful for a variety of applications. For example, the phosphopeptides are enzyme substrates which are modified (phosphorylated or dephosphorylated in response to different environmental cues provided to a cell. Identification of the enzymes for which the peptides are substrates, in turn, can be used to understand what intracellular signaling pathways are involved in any particular cellular response. To further illustrate, changes in phosphorylation states of substrate proteins can be used to identify kinases and/or phosphatases which are activated or inactivated in a manner dependent on particular cellular cues. In turn, those enzymes can be used as drug screening targets to find agents capable of altering their activity and, therefore, altering the response of the cell to particular environmental cues. So, for example, kinases and/or phosphatases which are activated in transformed (tumor) cells can be identified through their substrates, according to the subject method, and then used to develop anti-proliferative agents which are cytostatic or cytotoxic to the tumor cell.

In other embodiments, the present method can be used to identify a treatment that can modulate a modification of amino acid in a target protein without any knowledge of the upstream enzymes which produce the modified target protein. By comparing the level of phosphorylation of proteins including one or more of the subject phosphopeptide sequences before and after certain treatments, one can identify the specific treatment that leads to a desired change in level of modification to one or more target proteins. To illustrate, one can screen a library of compounds, for example, small chemical compounds from a library, for their ability to induce or inhibit phosphorylation of a target polypeptide. While in other instances, it may be desirable to screen compounds for their ability to induce or inhibit the dephosphorylation of a target polypeptide (i.e., by a phosphatase).

Similar treatments are not limited to small chemical compounds. For example, a large number of known growth factors, cytokines, hormones and any other known agents known to be able to modulate post-translational modifications are also within the scope of the invention.

In addition, treatments are not limited to chemicals. Many other environmental stimuli are also known to be able to cause post-translational modifications. For example, osmotic shock may activate the p38 subfamily of MAPK and induce the phosphorylation of a number of downtream targets. Stress, such as heat shock or cold shock, many activate the JNK/SAPK subfamily of MAPK and induce the phosphorylation of a number of downtream targets. Other treatments such as pH change may also stimulate signaling pathways characterized by post-translational modification of key signaling components.

In connection with those methods, the instant invention also provides a method for conducting a drug discovery business, comprising: i) by suitable methods mentioned above, determining the identity of a compound that modulates a modification of amino acid in a target polypeptide; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation.

The instant invention also provides a business method comprising: i) by suitable methods mentioned above, determining the identity of a compound that modulates a modification of amino acid in a target polypeptide; ii) licensing, to a third party, the rights for further drug development of compounds that alter the level of modification of the target polypeptide.

The instant invention also provides a business method comprising: i) by suitable methods mentioned above, determining the identity of the polypeptide and the nature of the modification induced by the treatment; ii) licensing, to a third party, the rights for further drug development of compounds that alter the level of modification of the polypeptide.

F. Exemplary Formulations

The subject compositions may be used alone, or as part of a conjoint therapy with other chemotherapeutic compounds.

The phosphopeptide therapeutics for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the phosphopeptide therapeutics, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the phosphopeptide therapeutics suitable for veterinary uses, e.g., for the treatment of live stock or domestic animals, e.g., dogs.

Other formulations of the present invention include agricultural formulations, e.g., for application to plants.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a phosphopeptide therapeutic at a particular target site.

The pharmaceutical compositions according to the present invention may be administered as either a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously. The pharmaceutical compositions of the present invention may be administered by any means that enables the phosphopeptide moiety to reach the targeted cells. In some embodiments, routes of administration include those selected from the group consisting of oral, intravesically, intravenous, intraarterial, intraperitoneal, local administration into the blood supply of the organ in which the targeted cells reside or directly into the cells. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastermal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, intravesically, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular phosphopeptide therapeutic employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebrovenitricular and subcutaneous doses of the compounds of this invention for a patient will range from about 0.0001 to about 100 mg per kilogram of body weight per day.

Because the subject ligands are specifically targeted to tumor bladder cells, those modified phosphopeptide which comprise chemotherapeutics or toxins can be administered in doses less than those which are used when the chemotherapeutics or toxins are administered as unconjugated active agents, preferably in doses that contain up to 100 times less active agent. In some embodiments, modified phosphopeptide which comprise chemotherapeutics or toxins are administered in doses that contain 10-100 times less active agent as an active moiety than the dosage of chemotherapeutics or toxins administered as unconjugated active agents. To determine the appropriate dose, the amount of compound is preferably measured in moles instead of by weight. In that way, the variable weight of different modified phosphopeptide does not affect the calculation. Presuming a one to one ratio of modified phosphopeptides to active moiety in modified phosphopeptides of the invention, less moles of modified phosphopeptide may be administered as compared to the moles of unmodified phosphopeptide administered, preferably up to 100 times less moles.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with other antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

Combined with certain formulations, the subject phosphopeptides can be effective intracellular agents. However, in order to increase the efficacy of Such phosphopeptides, the phosphopeptide can be provided a fusion peptide along with a second peptide which promotes “transcytosis”, e.g., uptake of the peptide by epithelial cells. To illustrate, the phosphopeptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the phosphopeptide can be provided a fusion polypeptide with all or a portion of the antenopedia III protein.

To further illustrate, the phosphopeptide (or peptidomimetic) can be provided as a chimeric peptide which includes a heterologous peptide sequence (“internalizing peptide”) which drives the translocation of an extracellular form of a phosphopeptide sequence across a cell membrane in order to facilitate intracellular localization of the phosphopeptide. In this regard, the therapeutic phosphopeptide sequence is one which is active intracellularly. The internalizing peptide, by itself, is capable of crossing a cellular membrane by, e.g., transcytosis, at a relatively high rate. The internalizing peptide is conjugated, e.g., as a fusion protein, to the phosphopeptide. The resulting chimeric peptide is transported into cells at a higher rate relative to the activator polypeptide alone to thereby provide an means for enhancing its introduction into cells to which it is applied, e.g., to enhance topical applications of the phosphopeptide.

In one embodiment, the internalizing peptide is derived from the Drosophila antennapedia protein, or homologs thereof. The 60 amino acid long long homeodomain of the homeo-protein antennapedia has been demonstrated to translocate through biological membranes and can facilitate the translocation of heterologous polypeptides to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193.

The present invention contemplates a phosphopeptide or peptidomimetic sequence as described herein, and at least a portion of the Antennapedia protein (or homolog thereof) sufficient to increase the transmembrane transport of the chimeric protein, relative to the phosphopeptide or peptidomimetic, by a statistically significant amount.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8).

Another exemplary transcellular polypeptide can be generated to include a sufficient portion of mastoparan (T. Higashijima et al., (1990) J. Biol. Chem. 265:14176) to increase the transmembrane transport of the chimeric protein.

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefor serve as an internalizing peptide for the subject transcellular peptides and peptidomimetics. Preferred growth factor-derived internalizing peptides include EGF (epidermal growth factor)-derived peptides, such as CMHIESLDSYTC (SEQ ID NO: 3) and CMYIEALDKYAC (SEQ ID NO: 4); TGF- beta (transforming growth factor beta )-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of phosphopeptides and peptidomimetics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

A particularly preferred pH-dependent membrane-binding internalizing peptide in this regard is aa1-aa2-aa3-EAALA(EALA)4-EALEALAA-amide (SEQ ID NO: 5), which represents a modification of the peptide sequence of Subbarao et al. (Biochemistry 26:2964, 1987). Within this peptide sequence, the first amino acid residue (aa1) is preferably a unique residue, such as cysteine or lysine, that facilitates chemical conjugation of the internalizing peptide to a targeting protein conjugate. Amino acid residues 2-3 may be selected to modulate the affinity of the internalizing peptide for different membranes. For instance, if both residues 2 and 3 are lys or arg, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore-forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

Mere membrane intercalation of an internalizing peptide may be sufficient for translocation of the phosphopeptide or peptidomimetic, across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

An exemplary accessory moiety in this regard would be a peptide substrate for N-myristoyl transferase, such as GNAAAARR (SEQ ID NO: 6, Eubanks et al., in: Peptides. Chemistry and Biology, Garland Marshall (ed.), ESCOM, Leiden, 1988, pp. 566-69) In this construct, an internalizing peptide would be attached to the C-terminus of the accessory peptide, since the N-terminal glycine is critical for the accessory moiety's activity. This hybrid peptide, upon attachment to an E2 peptide or peptidomimetic at its C-terminus, is N-myristylated and further anchored to the target cell membrane, e.g., it serves to increase the local concentration of the peptide at the cell membrane.

To further illustrate use of an accessory peptide, a phosphorylatable accessory peptide is first covalently attached to the C-terminus of an internalizing peptide and then incorporated into a fusion protein with a phosphopeptide or peptidomimetic. The peptide component of the fusion protein intercalates into the target cell plasma membrane and, as a result, the accessory peptide is translocated across the membrane and protrudes into the cytoplasm of the target cell. On the cytoplasmic side of the plasma membrane, the accessory peptide is phosphorylated by cellular kinases at neutral pH. Once phosphorylated, the accessory peptide acts to irreversibly anchor the fusion protein into the membrane. Localization to the cell surface membrane can enhance the translocation of the polypeptide into the cell cytoplasm.

Suitable accessory peptides include peptides that are kinase substrates, peptides that possess a single positive charge, and peptides that contain sequences which are glycosylated by membrane-bound glycotransferases. Accessory peptides that are glycosylated by membrane-bound glycotransferases may include the sequence x-NLT-x, where “x” may be another peptide, an amino acid, coupling agent or hydrophobic molecule, for example. When this hydrophobic tripeptide is incubated with microsomal vesicles, it crosses vesicular membranes, is glycosylated on the luminal side, and is entrapped within the vesicles due to its hydrophilicity (C. Hirschberg et al., (1987) Ann. Rev. Biochem. 56:63-87). Accessory peptides that contain the sequence x-NLT-x thus will enhance target cell retention of corresponding polypeptide.

In another embodiment of this aspect of the invention, an accessory peptide can be used to enhance interaction of the phosphopeptide or peptidomimetic with the target cell. Exemplary accessory peptides in this regard include peptides derived from cell adhesion proteins containing the sequence “RGD”, or peptides derived from laminin containing the sequence CDPGYIGSRC (SEQ ID NO: 7). Extracellular matrix glycoproteins, such as fibronectin and laminin, bind to cell surfaces through receptor-mediated processes. A tripeptide sequence, RGD, has been identified as necessary for binding to cell surface receptors. This sequence is present in fibronectin, vitronectin, C3bi of complement, von-Willebrand factor, EGF receptor, transforming growth factor beta, collagen type I, lambda receptor of E. Coli, fibrinogen and Sindbis coat protein (E. Ruoslahti, Ann. Rev. Biochem. 57:375-413, 1988). Cell surface receptors that recognize RGD sequences have been grouped into a superfamily of related proteins designated “integrins”. Binding of “RGD peptides” to cell surface integrins will promote cell-surface retention, and ultimately translocation, of the polypeptide.

As described above, the internalizing and accessory peptides can each, independently, be added to the phosphopeptide or peptidomimetic by either chemical cross-linking or in the form of a fusion protein. In the instance of fusion proteins, unstructured polypeptide linkers can be included between each of the peptide moieties.

In general, the internalization peptide will be sufficient to also direct export of the polypeptide. However, where an accessory peptide is provided, such as an RGD sequence, it may be necessary to include a secretion signal sequence to direct export of the fusion protein from its host cell. In preferred embodiments, the secretion signal sequence is located at the extreme N-terminus, and is (optionally) flanked by a proteolytic site between the secretion signal and the rest of the fusion protein.

In an exemplary embodiment, a phosphopeptide or peptidomimetic is engineered to include an integrin-binding RGD peptide/SV40 nuclear localization signal (see, for example Hart S L et al., 1994; J. Biol. Chem.,269:12468-12474), such as encoded by the nucleotide sequence provided in the Nde1-EcoR1 fragment: catatggutgactgccgtggcgatatgttcggttgcggtgctcctccaaaaaagaagagaaaggtagctggattc (SEQ ID NO: 8), which encodes the RGD/SV40 nucleotide sequence: MGGCRGDMFGCGAPPKKKRKVAGF (SEQ ID NO: 9). In another embodiment, the protein can be engineered with the HIV-1 tat(1-72) polypeptide, e.g., as provided by the Nde1-EcoR1 fragment: catatggagccagtagatcctagactagagccc-tggaagcatccaggaagtcagcctaaaactgcttgtaccaattgctattgtaaaaagtgttgctttcattgccaagtgtttc ataacaaaagcccttggcatctcctatggcaggaagaagcgagacagcgacgaagacctcctcaaggcagtcagact catcaagtttctctaagtaagcaaggattc (SEQ ID NO: 10), which encodes the HIV-1 tat(1-72) peptide sequence: MEPVDPRLEPWKHPGSQPKTACTNCYCKKCCFHCQVCFITKALGISYGRKK RRQRRRPPQGSQTHQVSLSKQ (SEQ ID NO: 11). In still another embodiment, the fusion protein includes the HSV-1 VP22 polypeptide (Elliott G., O'Hare P (1997) Cell, 88:223-233) provided by the Nde1-EcoR1 fragment:

(SEQ ID NO:12)
cat atg acc tct cgc cgc tcc gtg aag tcg ggt ccg cgg gag gtt ccg cgc gat
gag tac gag gat ctg tac tac acc ccg tct tca ggt atg gcg agt ccc gat agt
ccg cct gac acc tcc cgc cgt ggc gcc cta cag aca cgc tcg cgc cag agg
ggc gag gtc cgt ttc gtc cag tac gac gag tcg gat tat gcc ctc tac ggg ggc
tcg tca tcc gaa gac gac gaa cac ccg gag gtc ccc cgg acg cgg cgt ccc gtt
tcc ggg gcg gtt ttg tcc ggc ccg ggg cct gcg cgg gcg cct ccg cca ccc gct
ggg tcc gga ggg gcc gga cgc aca ccc acc acc gcc ccc cgg gcc ccc cga
acc cag cgg gtg gcg act aag gcc ccc gcg gcc ccg gcg gcg gag acc acc
cgc ggc agg aaa tcg gcc cag cca gaa tcc gcc gca ctc cca gac gcc ccc
gcg tcg acg gcg cca acc cga tcc aag aca ccc gcg cag ggg ctg gcc aga
aag ctg cac ttt agc acc gcc ccc cca aac ccc gac gcg cca tgg acc ccc cgg
gtg gcc ggc ttt aac aag cgc gtc ttc tgc gcc gcg gtc ggg cgc ctg gcg gcc
atg cat gcc cgg atg gcg gcg gtc cag ctc tgg gac atg tcg cgt ccg cgc aca
gac gaa gac ctc aac gaa ctc ctt ggc atc acc acc atc cgc gtg acg gtc tgc
gag ggc aaa aac ctg ctt cag cgc gcc aac gag ttg gtg aat cca gac gtg gtg
cag gac gtc gac gcg gcc acg gcg act cga ggg cgi tct gcg gcg tcg cgc
ccc acc gag cga cct cga gcc cca gcc cgc tcc gct tct cgc ccc aga cgg
ccc gtc gag gaa ttc

which encodes the HSV-1 VP22 peptide having the sequence:

(SEQ ID NO:13)

MTSRRSVKSGPREVPRDEYEDLYYTPSSGMASPDSPPDTSRRG
ALQTRSRQRGEVRFVQYDESDYALYGGSSSEDDEHPEVPRTR
RPVSGAVLSGPGPARAPPPPAGSGGAGRTPTTAPRAPRTGRVA
TKAPAAPAAETTRGRKSAQPESAALPDAPASTAPTRSKTPAQG
LARKLHFSTAPPNPDAPWTPRVAGFNKRVFCAAVGRLAAMH
ARMAAVQLWDMSRPRTDEDLNELLGUYTIRVTVCEGKNLLQR
ANELVNPDVVQDVDAATATRGRSAASRPTERPRAPARSASRP
RRPVE

In still another embodiment, the fusion protein includes the C-terminal domain of the VP22 protein from, e.g., the nucleotide sequence (Nde1-EcoR1 fragment):

(SEQ ID NO:14)
cat atg gac gtc gac gcg gcc acg gcg act cga ggg cgt tct gcg gcg tcg cgc ccc
acc gag cga cct cga gcc cca gcc cgc tcc gct tct cgc ccc aga cgg ccc gtc gag
gaa ttc

which encodes the VP22 (C-terminal domain) peptide sequence: MDVDAATATRGRSA-ASRPTERPRAPARSASRPRRPVE (SEQ ID NO: 15)

In certain instances, it may also be desirable to include a nuclear localization signal as part of the phosphopeptide.

In the generation of fusion polypeptides including the subject phosphopeptides, it may be necessary to include unstructured linkers in order to ensure proper folding of the various peptide domains. Many synthetic and natural linkers are known in the art and can be adapted for use in the present invention, including the (Gly₃Ser)₄ linker (SEQ ID NO: 2).

### EXAMPLE

Phosphoproteome Analysis by Mass Spectrometry

More than a 1,000 phosphopeptides were identified during the analysis of a whole cell lysate from S. cerevisiae. Sequences, including 383 sites of phosphorylation derived from 216 peptides were determined. Of these 60 were singly phosphorylated, 145 doubly phosphorylated, and 11 triply phosphorylated. To validate the approach, the results were compared with the literature, revealing 18 previously identified sites, including the doubly phosphorylated motif pTXpY derived from the activation loop of two MAP kinases. We note that the methodology can easily be extended to display and quantitate differential expression of phosphoproteins in two different cell systems, and therefore demonstrates an approach for “phosphoprofiling” as a measure of cellular states.

We prepared a standard mixture of tryptic peptides containing a single phosphopeptide and then analyzed the mixture before and after converting the peptides to the corresponding methyl esters. This rendered the IMAC selective for phosphopeptides, and eliminated confounding binding through carboxylate groups. Equimolar quantities of glyceraldehyde 3-phosphate dehydrogenase, bovine serum albumin, carbonic anydrase, ubiquitin, and β-lactoglobulin were digested with trypsin (approximately 125 predicted cleavage sites) and then combined with the phosphopeptide DRVpYIHPF (SEQ ID NO: 1, lower case p precedes a phosphorylated residue), to give a mixture that contained tryptic peptides at the 2 pmol/μl level and phosphopeptide at the 10 fmol/μl level. All experiments were performed on 0.5 μl aliquots of this solution.

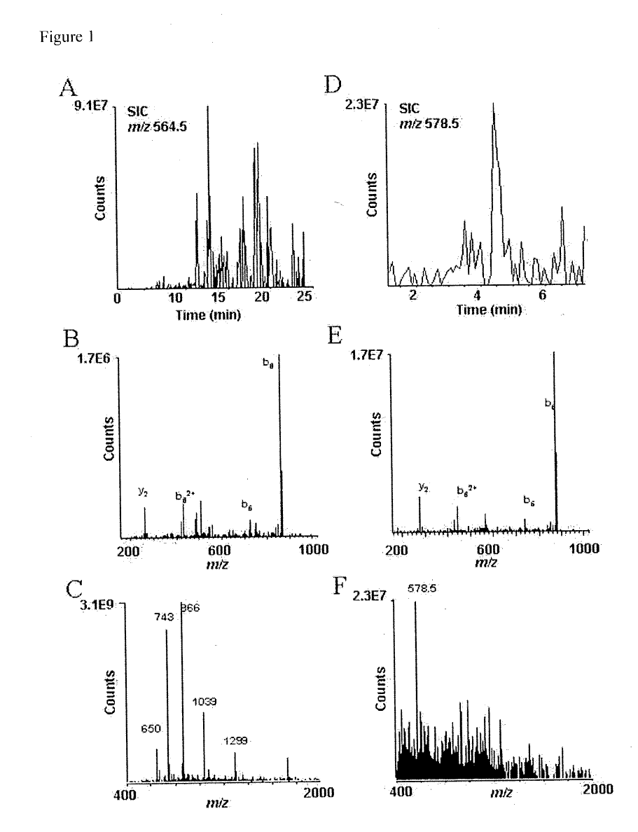

Shown in FIG. 1 are the results obtained when a 0.5 μl aliquot of the standard mixture was analyzed by a combination of IMAC^{5,6} and nanoflow-HPLC on the LCQ ion trap mass spectrometer. In this experiment, the instrument was set to cycle between two different scan functions every 2 sec throughout the HPLC gradient. Electrospray ionization spectra were recorded in the first of the two scans. MS/MS spectra on the (M+2H)⁺⁺ion of the phosphopeptide, DRVpYIHPF (SEQ ID NO: 1, m/z 564.5) were recorded in the second scan of the cycle. FIG. 1A shows a selected-ion-chromatogram (SIC) or plot of the ion current observed for m/z 564.5 as a function of scan number. Note that a signal at this m/z value is observed at numerous points in the chromatogram. Only ions at m/z 564.5 in scans 610-616 fragment to generate MS/MS spectra characteristic of the phosphopeptide, DRVpYIHPF (FIG. 1B). We conclude that DRVpYIHPF (SEQ ID NO: 1) elutes from the HPLC column in scans 610-616.

Displayed in FIG 1C is an electrospray ionization mass spectrum recorded during this same time period. Note that the spectrum contains signals of high intensity (ion currents of 1-3×10⁹) corresponding to nonphosphorylated tryptic peptides in the mixture but no signal above the chemical noise level for the phosphopeptide (m/z 564.5). We conclude that tryptic peptides containing multiple carboxylic acid groups can bind efficiently to the IMAC column, elute during the HPLC gradient, and suppress the signal from trace level phosphopeptides in the mixture.

To prevent binding of non-phosphorylated peptides to the IMAC column, all peptides in the standard mixture were converted to the corresponding peptide methyl esters and a 0.5 μl aliquot was then analyzed by the protocol outlined above. To detect the phosphopeptide in which both carboxylic acid groups had been esterified, MS/MS spectra were recorded on the (M+2H)⁺⁺ ion at m/z 578.5. The SIC for m/z 578.5 (FIG. 1D) suggests that the phosphopeptide dimethyl ester elutes during scans 151-163. Indeed, MS/MS spectra (FIG. 1E) recorded in this time window all contain the predicted fragments expected for the dimethyl ester of DRVpYIHPF (SEQ ID NO: 1). FIG. 1F shows an electrospray ionization mass spectrum recorded in the same area of the chromatogram (scan#154). Note that the parent ion, m/z 578.5 for the phosphopeptide dimethyl ester is now observed with a signal/noise of 3/1 and an ion current of 2×10⁷. This signal level on the LCQ is not atypical for phosphopeptpide samples at the 3-5 fmol level. Note also that signals above the chemical noise (ion current of 1×10⁷) for nonphosphorylated tryptic peptides no longer appear in this electrospray ionization spectrum or in any other spectrum recorded throughout the entire chromatogram. We conclude that conversion of carboxylic acid groups to methyl esters reduces nonspecific binding by at least two orders of magnitude and allows detection of phosphopeptides in complex mixtures down to the level of at least 5 fmol with the LCQ instrument.

To further evaluate the above protocol, we next analyzed a protein pellet (500 μg) obtained from a whole cell lysate of S. cerevisiae. If the average mol. wt. of yeast proteins is 25 kDa and half the genome is expressed and isolated in the pellet, then the average quantity each protein in the sample is expected to be approximately 5 pmol. If one makes the further assumption that 30% of expressed proteins contain at least one covalently bound phosphate, the total number of phosphoproteins in the sample could easily exceed 1,000. To evaluate this possibility, the pellet was digested with trypsin and the resulting peptides were converted to peptide methyl esters. One fifth of the resulting mixture was then fractionated by IMAC and analyzed by nano-flow HPLC on the LCQ ion trap mass spectrometer. Spectra were acquired with the instrument operating in the data-dependent mode throughout the HPLC gradient. Every 12-15 sec, the instrument cycled through acquisition of a full scan mass spectrum and 5 MS/MS spectra recorded sequentially on the 5 most abundant ions present in the initial MS scan. More than 1,500 MS/MS spectra were recorded in this mode of operation during the chromatographic separation.

Data acquired in the above experiment were analyzed both by a computer algorithm, the Neutral Loss Tool, and also by SEQUEST. The Neutral Loss Tool searches MS/MS spectra for fragment ions formed by loss of phosphoric acid, 32.6, 49 or 98 Da from the (M+3H)⁺⁺⁺, (M+2H)⁺⁺ and (M+H)⁺ ions, respectively. Phosphoserine and phosphothreonine, but not phosphotyrosine, lose phosphoric acid readily during the collision activation dissociation process in the ion trap mass spectrometer. Thus, appearance of fragment ions 32.6, 49 or 98 Da below the triply, doubly or singly charged precursor ions in peptide MS/MS spectra strongly suggests that the peptide contains at least one phosphoserine or phosphothreonine residue. In the above experiment, more than 1,000 different phosphoserine or phosphothreonine containing peptides were detected in the yeast whole cell lysate with the Neutral Loss Tool.

To identify phosphopeptides in the above sample, MS/MS spectra were searched with the SEQUEST algorithm against yeast protein database (obtained from the Saccharomyces Genome Database (SGD) http://genome-www.stanford.edu/Saccharomyces/). Of the 216 sequence confirmed, 60 (28%) are singly phosphorylated, 145 (67%) are doubly phosphorylated, and 11 (5%) are triply phosphorylated.

Phosphorylation sites that were previously identified are in bold face in the table. Two of the sequences derive from mitogen-activated protein (MAP) kinases encoded by HOG1 and SLT2 and contain the doubly phosphorylated motif pTXpY derived from the activation loop in their respective catalytic domains. This clearly indicates the potential of the phosphoprofiling approach as a measure of cellular activation states. In fact, the list contains 171 different proteins, including abundant species such as the heat shock proteins (Hsp26p, Hsp30p) and those involved in carbohydrate metabolism (Hxk1p, Hxt2p), and protein synthesis (Rp112AP, Rp124Ap). Rare proteins such as the cell cycle regulatory molecules, Mob1p and Ash1p, and cytoplasmic proteins such as Myo3p and Pea2p, also appear in the Table. Of the 216 peptides in Table 1, 66 have sequences that correspond to a codon bias of less than 0.1 and are therefore likely to be expressed in low copy number 11.

Eighty-five additional phosphopeptides were identified by recording MS/MS on the sample eluted from the IMAC column after it had been treated with alkaline phosphatase to remove covalently bound phosphate. In this experiment, peptide methyl esters were eluted from the IMAC column directly to a second column packed with F7m Polyvinyl spheres containing immobilized alkaline phosphatase. Dephosphorylated peptides were then eluted to the standard nano-flow HPLC column and analyzed on the LCQ using the data dependent scan protocol described above. This approach has the advantage that the resulting MS/MS spectra usually contain a larger number of abundant, sequence-dependent, fragment ions than those recorded on the corresponding phosphorylated analogs. This, in turn, improves the likelihood that the SEQUEST algorithm will find a unique match in the protein database. The disadvantage of the protocol is that the resulting MS/MS spectra no longer contain information on the number and location of the phosphorylated residues within the peptide.

Methods

Protein extraction from S. cerevisiae. Yeast strain 2124 MATa ade2-1, ade6-1, leu2-3,112, ura3-52, his3Δ1, trp1-289, can1 cyh2 bar1::KAN (40 ml) was grown in YPD at 23° C. to a density of 1×10⁷ cells/ml. The cell pellet was resuspended in 1.5 ml of Trizol (Gibco-BRL) and cell lysis was performed by homogenization with glass beads in 3 consecutive sessions of 45 sec each in a Fastprep FP120 shaker (Savant). Total yeast protein, free of nucleic acids, was extracted from this yeast lysate using Trizol according to the manufacturer's directions (Gibco-BRL). The protein pellet was resuspended in 1% SDS, and dialyzed against 1% SDS using a Slyde-A-Lyzer, 10,000 MW cutoff (Pierce) to remove small molecules and stored at −80° C. To follow the removal of nucleotide, 0.1 μl of a P³² CTP (Amersham-Pharmacia) was added to a 10 ml equivalent of lysed cells. Aliquots were removed after each step in purification and the amount of nucleotide was quantitated by Scintillation with Scintisafe EconoF (Fischer). Yeast protein, 500 μg (approximately 10 nmol), in 500 μl of 100 mM ammonium acetate (pH 8.9), was digested with trypsin (20 μg) (Promega) overnight at 37° C. Solvent was removed by lyophilization and the residue was reconstituted in 400 μl of 2N methanolic HCl and allowed to stand at room temperature for 2 h. Solvent was lyophilized and the resulting peptide methyl esters were dissolved in 120 μl of a solution containing equal parts of methanol, water and acetonitrile. An aliquot corresponding to 20% of this material (2 nmol of yeast protein) was subjected to chromatography and mass spectrometry as described below.

Chromatography. Construction of immobilized metal affinity chromatography (IMAC) columns has been described previously⁹. Briefly, 360 μm O.D.×100 μm I.D. fused silica (Polymicro Technologies, Phoenix, Ariz.) was packed with 8 cm POROS 20 MC (PerSeptive Biosystems, Framingham, Mass.). Columns were activated with 200 μl 100 mM FeCl₃ (Aldrich, Milwaukee, Wis. and loaded with either 0.5 μl of the above standard mixture or sample corresponding to peptides derived from 100 μg (10 nmol) of protein extract from S. cerevisiae. To remove non-specific binding peptides, the column was washed with a solution containing 100 mM NaCl (Aldrich) in acetonitrile (Mallinkrodt, Paris, Ky.), water, and glacial acetic acid (Aldrich) (25:74:1, v/v/v). For sample analysis by mass spectrometry, the affinity column was connected to a fused silica pre-column (6 cm of 360 μm O.D.×100 μm I.D.) packed with 5-20 μm C]8 particles (YMC, Wilmington, N.C.). All column connections were made with 1 cm of 0.012″ I.D.×0.060″ O.D. Teflon tubing (Zeus, Orangeburg, S.C.). Phosphopeptides were eluted to the pre-column with 10 μl 50 mM Na₂HPO₄ (Aldrich) (pH 9.0) and the pre-column was then rinsed with several column volumes of 0.1% acetic acid to remove Na₂HPO₄. The pre-column was connected to the analytical HPLC column (360 μm O.D.×100 μm I.D. fused silica) packed with 6-8 cm of 5 μm C18 particles (YMC, Wilmington, N.C.). One end of this column contained an integrated laser pulled ESI emitter tip (2-4 μm in diameter)¹⁴. Sample elution from the HPLC column to the mass spectrometer was accomplished with a gradient consisting of 0.1% acetic acid and acetonitrile. For removal of phosphate from the tryptic peptides, the IMAC column was connected to a fritted 360 μm O.D.×200 μm I.D. fused silica capillary packed with F7m (Polyvinyl spheres), containing immobilized alkaline phosphatase (MoBiTech, Marco Island, Fla.). Phosphopeptides were eluted from the IMAC column through the phosphatase column onto a precolumn with 25 μL of 1 mM ethylenediaminetetraacetic acid (EDTA) (pH 9.0), and the pre-column was then rinsed with several column volumes of 0.1% acetic acid to remove EDTA. The pre-column was connected to an analytical HPLC column. Sample elution from the HPLC column to the mass spectrometer was accomplished with a gradient consisting of 0.1% acetic acid and acetonitrile.

Mass Spectrometry. All samples were analyzed by nanoflow-HPLC/microelectrospray ionization on a Finnigan LCQ® ion trap (San lose, Calif.). A gradient consisting of 0-40% B in 60 min. 40-100% B in 5 min (A=100 mM acetic acid in water, B=70% acetonitrile, 100 mM acetic acid in water) flowing at approximately 10 nL/min was used to elute peptides from the reverse-phase column to the mass spectrometer through an integrated electrospray emitter tip¹⁴. Spectra were acquired with the instrument operating in the data-dependent mode throughout the HPLC gradient. Every 12-15 sec, the instrument cycled through acquisition of a full scan mass spectrum and 5 MS/MS spectra (3 Da window; precursor m/z ±1.5 Da, collision energy set to 40%, dynamic exclusion time of 1 minute) recorded sequentially on the 5 most abundant ions present in the initial MS scan. To perform targeted analysis of the phosphopeptide in the standard mixture, the ion trap mass spectrometer was set to repeat a cycle consisting of a full MS scan followed by an MS/MS scan (collision energy set to 40%) on the (M+2H)⁺⁺ of DRVpYIHPF (SEQ ID NO: 1) or its methyl ester (m/z 564.5 and 578.5, respectively). The gradient employed for this experiment was 0-100% B. in 30 minutes for the underivatized sample, 0-100% B in 17 minutes for derivatized sample (A=100 mM acetic acid in water, B=70% acetonitrile, 100 mM acetic acid in water).

Database Analysis. All MS/MS spectra recorded on tryptic phosphopeptides derived from the yeast protein extract were searched against the S. cerevisiae protein database by using the SEQUEST algorithms¹⁰. Search parameters included a differential modification of +80 Da (presence or absence of phosphate) on serine, threonine and tyrosine and a static modification of +14 Da (methyl groups) on aspartic acid, glutamic acid, and the C-terminus of each peptide.

TABLE 1
Phosphorylated peptide sequences from S. cerevisiae

| Protein | Phosphopeptide Sequence | Protein | Phosphopeptide Sequence |
|---|---|---|---|
| GPD2 | SDpSAVpSIVHLK | TIF5 | AAKPFITWLETAEpSDDDEEDDE |
| | (SEQ ID NO:16) | | (SEQ ID NO:123) |
| GPD2 | SDpSAVSIVHLK | TPO1, YLL028W | TTTMNpSAAEpSEVNITR |
| | (SEQ ID NO:17) | | (SEQ ID NO:124) |
| GSY1 | IARPLpSVPGpSPK | TPS2 | SApSYTGAKV |
| | (SEQ ID NO:18) | | (SEQ ID NO:125) |
| GSY2 | VARPLpSVPGpSPR | TPS3 | TSpSSMpSVGNNK |
| | (SEQ ID NO:19) | | (SEQ ID NO:126) |
| GTS1 | SHpSFYK | TR5120 | ATASTpTASSpTPR |
| | (SEQ ID NO:20) | | (SEQ ID NO:127) |
| HOG1 | IQDPQMpTGpYVSTR | TSL1 | SApTRpSPSAFNR |
| | (SEQ ID NO:21) | | (SEQ ID NO:128) |
| HSP26 | KIEVSpSQEpSWGN | TYS1 | GYPVApTPQK |
| | (SEQ ID NO:22) | | (SEQ ID NO:129) |
| HSP26 | KIEVSSQEpSWGN | UBP1 | AAQQDpSpSEDENIGGEYYTK |
| | (SEQ ID NO:23) | | (SEQ ID:130) |
| HSP26 | QLANpTPAK | UGP1 | THpSTYAFESNTNSVAASQMR |
| | (SEQ ID NO:24) | | (SEQ ID:131) |
| HSP30 | EAVPEpSPR | VPS13 | TApTPQpSLQGSNIC |
| | (SEQ ID NO:25) | | (SEQ ID NO:132) |
| HTA1 | ATKApSQEL | VRP1 | NPTKpSPPPPPpSPSTMDTGTSNpSPSK |
| | (SEQ ID NO:26) | | (SEQ ID NO:133) |
| HXK1/HXK2 | KGpSMADVPK | VRP1 | SPPPPPpSPSTMDTGTSNpSPSK |
| | (SEQ ID NO:27) | | (SEQ ID NO:134) |
| HXT2 | LEpTDEpSPIQTK | YAK1 | RApSLNSK |
| | (SEQ ID NO:28) | | (SEQ ID NO:135) |
| HXT2 | VESGpSQQTpSIHpSTPIVQK | YAK1 | RKpSpSLVVPPAR |
| | (SEQ ID NO:29) | | (SEQ ID NO:136) |
| HXT2 | VESGSQQTpSIHpSTPIVQK | YDR235W | pSQNNLQK |
| | (SEQ ID NO:30) | | (SEQ ID NO:137) |
| IRA2 | NSDNVNpSLNSpSPK | YBT1 | SSILpSRANpSSANLAAK |
| | (SEQ ID NO:31) | | (SEQ ID NO:138) |
| IST2 | TTESSSpSSpSAAK | YDT1 | ETSNEASpSpTNSENVNK |
| | (SEQ ID NO:32) | | (SEQ ID NO:139) |
| IST2 | VPpTVGpSYGVAGATLPETIPTSK | YCR023C | SpSLSpSLSNQR |
| | (SEQ ID NO:33) | | (SEQ ID NO:140) |
| KSP1 | MEGGDNESpSSTpSPDER | YDL113C | NGVGpSPKKpSPK |
| | (SEQ ID NO:34) | | (SEQ ID NO:141) |
| MCM3 | RSTASpSVNATPpSSARR | YDL166C | MWLEQHPDGVTNEYQGPRpSDDEDDED |
| | (SEQ ID NO:35) | pSE | |
| | | | (SEQ ID NO:142) |
| MCM3 | VRQPApSNSGpSPIK | YDL189W | RApS VEGpSPSSR |
| | (SEQ ID NO:36) | | (SEQ ID NO:143) |
| MLE3 | TTELPATpSPYVpSPQQSAR | YDL222C | AHSTYSDHDMYAQYESPpSVDpTGAQM |
| | (SEQ ID NO:37) | EK | |
| | | | (SEQ ID NO:144) |
| MOB1 | MpSPVLTpTPKR | YDL223C | GSDYDYNNSTHpSAEHpTPR |
| | (SEQ ID NO:38) | | (SEQ ID NO:145) |
| MON2, | NIpSTSpSVTTSPVESTK | YDL223C | KVSVGpSMGpSGK |
| YNL297C | (SEQ ID NO:39) | | (SEQ ID NO:146) |
| MRH1 | APVApSPRPAApTPNLSK | YDR090C | YSRLpSV |
| | (SEQ ID NO:40) | | (SEQ ID NO:147) |
| MSC3 | STAGNNNDpSRANpSITVK | YDR262W | NVpSRTpSPNTpThTK |
| | (SEQ ID NO:41) | | (SEQ ID NO:148) |
| MSL5 | NRpSPpSPPPVYDAQOK | YDR262W | NVpSRTpSPNTTNTK |
| | (SEQ ID NO:42) | | (SEQ ID NO:149) |
| MSN2 | RPpSYR | YDR372C | ADpSGDTSpSIHSSANNTK |
| | (SEQ ID NO:43) | | (SEQ ID NO:150) |
| MYO3 and 5 | SMpSLLGYR | YDR372C | ADSGDTSpSIIISpSANNTK |
| | (SEQ ID NO:44) | | (SEQ ID NO:151) |
| NCB2 | LHHNpSVSDPVKSEDS*pS | YDR384C | TpSSApSpSPQDLEK |
| | (SEQ ID NO:45) | | (SEQ ID NO:152) |
| NCB2 | LHHNSVSDPVKpSEDS*pS | YDR466W | ApSSEPSpSPPPISR |
| | (SEQ ID NO:46) | | (SEQ ID NO:153) |
| NEW1 | GTPKPVDpTDDEED | YEF3 | KKELGDAYVpSpSDEEF |
| | (SEQ ID NO:47) | | (SEQ ID NO:154) |
| NIP1 | SSNYDpSpSDEEpSDEEDGKK | YFR016C | SKTPEpSPK |
| | (SEQ ID NO:48) | | (SEQ ID NO:155) |
| NOT3 | IGpSALNpTPK | YFR017C | RRpSTNYMDALNSR |
| | (SEQ ID NO:49) | | (SEQ ID NO:156) |
| NOT3 | TPTTAAATTTSpSNANpSR | YFR017C | RSpSGPMDFQNTIHNMQYR |
| | (SEQ ID NO:50) | | (SEQ ID NO:157) |
| NPL3 | GGYDpSPR | YFR024C | LAPTNpSGGpSGGKLDDPSGASSYYASHR |
| | (SEQ ID NO:231) | | (SEQ ID NO:158) |
| NPR1 | QSpSIYSASR | YGR138C | LTKpTEpTVK |
| | (SEQ ID NO:51) | | (SEQ ID NO:159) |
| NRD1 | NRpSRpSPPAPESQPSTGR | YGR138c | TApSALpSR |
| | (SEQ ID NO:52) | | (SEQ ID NO:160) |
| NTH1 | RGpSEDDTYSSSQGNR | YHR052W | SNpSKKSpTPVpSTPSKEK |
| | (SEQ ID NO:53) | | (SEQ ID NO:161) |
| NTH1 | RLSpSLpSEFNDPFSNAEVYYGPPTDPR | YHR052W | SNpSKKSpTPVSTPSK |
| | (SEQ ID NO:54) | | (SEQ ID NO:162) |
| NUP145 | AYEPDLpSDADFEGIEApSPK | YHR097C | ANSpSTpTTLDAIKPNSK |
| | (SEQ ID NO:55) | | (SEQ ID NO:163) |
| NUP2 | EpTYDpSNEpSDDDVTPSTK | YHR132W-A | RMpSpSSSGGDSISR |
| | (SEQ ID NO:56) | | (SEQ ID NO:164) |
| NUP2 | ETYDpSNEpSDDDVTPSTK | YHR186C | AGpSIQpTQSR |
| | (SEQ ID NO:57) | | (SEQ ID NO:165) |
| ABF1 | SNpSIDYAK | REG1, YDR028C | SGSpTNpSLYDLAQPSLSSATPQQK |
| | (SEQ ID NO:58) | | (SEQ ID NO:166) |
| ABP1 | KEPVKpTPpSPAPAAK | RPA 190 | DKEpSDpSDpSEDEDVDMNEQINK |
| | (SEQ ID NO:59) | | (SEQ ID NO:167) |
| ABP1 | SFpTPSKpSPAPVSK | RPL12A | IGPLOLpSPK |
| | (SEQ ID NO:60) | | (SEQ ID NO:168) |
| ACC1 | AVpSVSDLSYVANSQSSPLR | RPL24A | VAATpSR |
| | (SEQ ID NO:61) | | (SEQ ID NO:169) |
| ACE2 | KLFpSPK | RPL25 | TpSATFR |
| | (SEQ ID NO:62) | | (SEQ ID NO:170) |
| AKL1 | DKDpSNSpSITISTSTPSEMR | RPL3 | AApSIR |
| | (SEQ ID NO:63) | | (SEQ ID NO:171) |
| AKL1 | STSpSYSSGGR | RPL7A, RPL7B | ILpTPEpSQLKK |
| | (SEQ ID NO:64) | | (SEQ ID NO:172) |
| BFR2, | NGEpSDLpSDYONSNTEETK | RPN8 | VpSDDSEpSESGDKEATAPLIQR |
| YDR299W | (SEQ ID NO:65) | | (SEQ ID NO:173) |
| BLM3 | pSApTPTLQDQK | RPP1A and 2B | (E)EEAKEEpSDDDMGFGLPD |
| | (SEQ ID NO:66) | | (SEQ ID NO:174) |
| BN15 | pSGGSpTPLDSQTK | RPS31 | VYTpTPKK |
| | (SEQ ID NO:67) | | (SEQ ID NO:175) |
| BN15 | YDpSPVSpSPITSASELGSIAK | RPS6A | ASpSLKA |
| | (SEQ ID NO:68) | | (SEQ ID NO:176) |
| B012 | ALpSPIPSPpTR | RPS6A | RApSpSLKA |
| | (SEQ ID NO:69) | | (SEQ ID NO:177) |
| BRE5 | ESGNNASpTPSpSSPEPVANPPK | SEC1 | SQDNSPKpSGTSpSPK |
| | (SEQ ID NO:70) | | (SEQ ID NO:178) |
| BUD4 | VNpSELEEpSPAAVHQER | SEC3 | TIpSGSpSAI-1I-ISR |
| | (SEQ ID NO:71) | | (SEQ ID NO:179) |
| CCC1 | HNDLpSSSpSSDIIYGR | SEC31 | APpSSVPSMVSPPPLHK |
| | (SEQ ID NO:72) | | (SEQ ID NO:180) |
| CDC11 | LNGpSSSpSSTTTR | SEC31 | APpSSVSMVpSPPPLHK |
| | (SEQ ID NO:73) | | (SEQ ID NO:181) |
| CDC39 | RQpTPLQSNA | SEC31 | VPpSLVATSEpSPR |
| | (SEQ ID NO:74) | | (SEQ ID NO:182) |
| CDC47 | FVDDGTMDpTDQEDSLVpSTPK | SEC31 | VPSLVATSEpSPR |
| | (SEQ ID NO:75) | | (SEQ ID NO:183) |
| CHD1 | NSVNGDGTAANpSDpSDDDSTSR | SEC4 | EGNIpSINpSGSGNS |
| | (SEQ ID NO:76) | | (SEQ ID NO:184) |
| CHO1, YER026C | DENDGYApSDEVGGTLpSR | SEC4 | TVpSASpSGNGK |
| | (SEQ ID NO:77) | | (SEQ ID NO:185) |
| CHS2 | TQFYRDpSAHNpSPVAPNR | SGV1 | YpTSVVVTR |
| | (SEQ ID NO:78) | | (SEQ ID NO:186) |
| CLA4 | GPMHPNNpSQRpSLQQQQQQQQQQK | SHP1 | KGpSTpSPEPTK |
| | (SEQ ID NO:79) | | (SEQ ID NO:187) |
| CRN1 | QEAPKpSPpSPLK | SIN3 | VpTTPMGTTTVNNNIpSPSGR |
| | (SEQ ID NO:80) | | (SEQ ID NO:188) |
| CRN1 | TKpSPEQEKSApTPPSSITAAK | SLA2 | TPpTPTPPVVAEPAIpSPRPVSQR |
| | (SEQ ID NO:81) | | (SEQ ID NO:189) |
| CTK1 | ADYpTNR | SLT2 | GYSENPVENSQFLpTEpYVATR |
| | (SEQ ID NO:82) | | (SEQ ID NO:190) |
| CTK3 | DSITSpSSTpTTPPSSQQK | SMI1 | SQQGLSHVTSTGpSSSpSMER |
| | (SEQ ID NO:83) | | (SEQ ID NO:191) |
| CYK3 | LpSSSMPNpSPKKPVDSLTK | SMY2 | SQFQKpSPK |
| | (SEQ ID NO:84) | | (SEQ ID NO:192) |
| DBP10 | LQNSNNEADpSDpSDDENDR | SOK2, YIL05SC | SIpSPR |
| | (SEQ ID NO:85) | | (SEQ ID NO:193) |
| DIP5 | MFTSTpSPR | SOL1 | STQMpSOTpSLNONONTESK |
| | (SEQ ID NO:86) | | (SEQ ID NO:194) |
| DIP5 | NSpSSLDpSDHDAYYSK | SOL1 | VNpSVRpSNASSR |
| | (SEQ ID NO:87) | | (SEQ ID NO:195) |
| ECM25 | SRpSPSPQR | SOL2 | SpTApSAAEOK |
| | (SEQ ID NO:88) | | (SEQ ID NO:196) |
| EDE1 | GVATpTPK | SPC98 | pSMVpSSPNR |
| | (SEQ ID NO:89) | | (SEQ ID NO:197) |
| EDE1 | RANSNEDDGEpSVpSSIQEpSPK | SRA1 | SRpSSVMFK |
| | (SEQ ID NO:90) | | (SEQ ID NO:198) |
| EDE1 | TTPLpSANSpTGVSSLTR | SSD1 | SSpTINNDSDSLSpSPTK |
| | (SEQ ID NO:91) | | (SEQ ID NO:199) |
| ENO1, ENO2 | pSVYDSR | STE2 | EGEVEPVDMYpTPDpTAADEEAR |
| | (SEQ ID NO:92) | | (SEQ ID NO:200) |
| ERG6 | VARKPENAETPpSQTpSQEATQ | STE2 | YQLPpTPpTpSSKNTR |
| | (SEQ ID NO:93) | | (SEQ ID NO:201) |
| FEN1 | NVPpTPpSPpSPKPQHR | STF2 | RGpSNLQSHEQK |
| | (SEQ ID NO:94) | | (SEQ ID NO:202) |
| FPR4 | LEEDEpSEPSEQEADVPKR | SU12 | ELDNRpSDpSEDDEDEpSDDE |
| | (SEQ ID NO:95) | | (SEQ ID NO:203) |
| GCS1 | SApTPANSpSNGANFQK | SUR7 | pSHERPDDVpSV |
| | (SEQ ID NO:96) | | (SEQ ID NO:204) |
| GLY1 | SESpTEVDVDGNAIR | SUR7 | SHERPDDVpSV |
| | (SEQ ID NO:97) | | (SEQ ID NO:205) |
| GNP1 | KSpSYIpTVDGIK | SW14 | STpSETSpSPK |
| | (SEQ ID NO:98) | | (SEQ ID NO:206) |
| GPA2 | NGSTPDTQTApSAGpSDNVOK | SYG1 | RRpSpSVFENISR |
| | (SEQ ID NO:99) | | (SEQ ID NO:207) |
| GPD1 | SSpSSVpSLK | TAT1 | QDEVpSGQpTAEPR |
| | (SEQ ID NO:100) | | (SEQ ID NO:208) |
| OAF1 | SApSPINTNNASGDpSPDTKK | YHR186C | FAVANLpSTMpSLVNNPALQSR |
| | (SEQ ID NO:101) | | (SEQ ID NO:209) |
| S45866 | AHNVpSTSNNSPpSTDNDSISK | YML029W | SQpSPVpSFAPTQGR |
| | (SEQ ID NO:102) | | (SEQ ID NO:210) |
| S45866 | SYpTNTTKPK | YML072C | ASpSFAR |
| | (SEQ ID NO:103) | | (SEQ ID NO:211) |
| PAM1 | SDQGNNpSpSONDSR | YML072C | SPpSNLNSTSVpIPR |
| | (SEQ ID NO:104) | | (SEQ ID NO:212) |
| PAN1 | DASApSSTSpTFDAR | YMR196W | IGGTHSGLpTPQSSISpSDKAR |
| | (SEQ ID NO:105) | | (SEQ ID NO:213) |
| PAN1 | SSpSPSYSQFK | YMR295C | SSIpSNTpSDHDGANR |
| | (SEQ ID NO:106) | | (SEQ ID NO:214) |
| PAT1 | RRpSSpYAFNNONGATNLNK | YNL136W | HSSSTONTpSNETpSPK |
| | (SEQ ID NO:107) | | (SEQ ID NO:215) |
| PBS2 | SASVGpSNQSEQDKGSSQpSPK | YNLI56C | ILpSASpSIHENFPSR |
| | (SEQ ID NO:108) | | (SEQ ID NO:216) |
| PDA1 | YGGHpSMSDPOTTYR | YNL156C | SVpSIDpSTK |
| | (SEQ ID NO:109) | | (SEQ ID NO:217) |
| PDR5 | TLTAQSMQNpSTQpSAPNK | YNL321W | SHApSVPDLNTATPpSSPKR |
| | (SEQ ID NO:110) | | (SEQ ID NO:218) |
| PEA2 | NTSpSPPIpSPNAAAIQEEDSSK | YOR042W | VVAETTYIDpTPDpTETKKK |
| | (SEQ ID NO:111) | | (SEQ ID NO:219) |
| PFK2 | VHpSYTDEAYR | YOR052C | SSpSNSpSVTSTGQSSR |
| | (SEQ ID NO:112) | | (SEQ ID NO:220) |
| PMA1, PMA2 | VSpTQIIEK | YOR175C | KMSFpSGYpSPKPISK |
| | (SEQ ID NO:113) | | (SEQ ID NO:221) |
| POM34 | YAYMMNpSQpSPR | YOR220W | GGSSLpSPDKSSLEpSPTMLK |
| | (SEQ ID NO:114) | | (SEQ ID NO:222) |
| POM34 | YAYMMNSQpSPR | Y0R273C | TMEpTDPpSTR |
| | (SEQ ID NO:115) | | (SEQ ID NO:223) |
| PRS5 | KTTpSTSpSTpSSQSSNSSK | YPE247C | SpSISFGSSQR |
| | (SEQ ID NO:116) | | (SEQ ID NO:224) |
| PRS5 | TTpSTSpSTSSQSSNSSK | YPR156C | TEpTVKpSEQDMGVSSK |
| | (SEQ ID NO:117) | | (SEQ ID NO:225) |
| PTR2 | ANDIEIEEPMEpSLRpSTTKY | YPR156C | TSpTAIpSR |
| | (SEQ ID NO:118) | | (SEQ ID NO:226) |
| PTR2 | DSYVpSDDVANpSTER | YRO2 | KAQEEEEDVApTDpSE |
| | (SEQ ID NO:119) | | (SEQ ID NO:227) |
| RAS2 | KMpSNAANGK | Y5C84 | GYGDFDpSEDEDYDYGR |
| | (SEQ ID NO:120) | | (SEQ ID NO:228) |
| RAS2 | NApSIEpSKTGEAGNQATNOK | ZUO1 | NHTWpSEFER |
| | (SEQ ID NO:121) | | (SEQ ID NO:229) |
| REG1 | RpSDSOVHpSPITDNSSVASSTTSR | TyB protein | TDSSpSADpSDMTSTKKY |
| | (SEQ ID NO:122) | | (SEQ ID NO:230) |

References:

1. Hubbard, M. J. and Cohen, P. On target with a new mechanism for the regulation of protein phosphorylation. Trends Biochem. Sci. 18, 172-177 (1993).

2. Annan, R., Huddleston, M., Verma, R., Deshaies, R. & Carr, S. A Multidimensional Electrospray MS-Based Approach to Phosphopeptide Mapping. Anal. Chem. 73, 393-404 (2001).

3. Oda, Y., Nagasu, T. & Chait, B. Enrichment analysis of phosphorylated proteins as a tool for probing the phosphoproteome. Nat. Biotechnol. 19, 379-382 (2001).

4. Zhou, H., Watts, J. & Aebersold, R. A systematic approach to the analysis of protein phosphorylation. Nat. Biotechnol. 19, 375-378 (2001).

5. Andersson, L. and Porath, J. Isolation of phosphoproteins by immobilized metal (Fe3+) affinity chromatography. Anal. Biochem. 154, 250-254 (1986b).

6. Michel, H., Hunt, D. F., Shabanowitz, J. and Bennett, J. Tandem mass spectrometry reveals that three photosystem II proteins of spinach chloroplasts contain N-acetyl-O-phosphothreonine at their NH₂ termini. J. Biol. Chem. 263, 1123-1130 (1988).

7. Muszynska, G., Dobrowolska, G., Medin, A., Ekman, P. & Porath, J. O. Model studies on iron(III) ion affinity chromatography. II. Interaction of immobilized iron(III) ions with phosphorylated amino acids, peptides and proteins. J. Chrom. 604, 19-28 (1992).

8. Nuwaysir, L. & Stults, J. Electrospray ionization mass spectrometry of phosphopeptides isolated by on-line immobilized metal-ion affinity chromatography. J. Amer. Soc. Mass Spectrom. 4, 662-669 (1993).

9. Zarling, A. L. et al. Phosphorylated peptides are naturally processed and presented by major histocompatibility complex class I molecules in vivo. J. Exp. Med. 192, 1755-1762 (2000).

10. Eng, J., McCormack, A. L. and Yates, J. R. An approach to correlate tandem mass spectral data of peptides with amino acid sequences in a protein database. J. Amer. Soc. Mass Spectrom, 5, 976-989 (1994).

11. Bennetzen, J. L. & Hall, B. D. Codon selection in yeast. J Biol Chem 257, 3026-3031 (1982).

12. Zhang, X. et al. Identification of phosphorylation sites in proteins separated by polyacrylamide gel electrophoresis. Anal Chem 70, 2050-2059 (1998).

13. Amankwa, L. N., Harder, K., Jirik, F. & Aebersold, R. High-sensitivity determination of tyrosine-phosphorylated peptides by on-line enzyme reactor and electrospray ionization mass spectrometry. Prot. Sci. 4, 113-125 (1995).

14. Martin, S. E., Shabanowitz, J., Hunt, D. F. & Marto, J. A. Subfemtomole ms and ms/ms peptide sequence analysis using nano-hplc micro-esi fourier transform ion cyclotron resonance mass spectrometry. Anal Chem 72, 4266-4274 (2000).

We Claim:

1. A peptide of 4-20 amino acids in length including one or more phosphopeptide sequences shown in Table 1, or corresponding phosphopeptide sequence(s) of a homologous mammalian protein.

2. An isolated or recombinant polypeptide which includes one or more phosphopeptide sequences shown in Table 1, or corresponding phosphopeptide sequence(s) of a homologous mammalian protein.

3. A peptidomimetic including a phosphopeptide sequences shown in Table 1, or corresponding phosphopeptide sequence(s) of a homologous mammalian protein, having one or more peptide bond replacements or non-naturally occurring amino acid sidechains, wherein the peptidomimetic.

4. The peptide of claim 1, polypeptide of claim 2 or peptidomimetic of claim 3, including at least one phosphorylated amino acid residue or analog of a phosphorylated amino acid residue.

5. The peptide of claim 1, polypeptide of claim 2 or peptidomimetic of claim 3, wherein the phosphopeptide sequence mediates binding to at least one of a kinase, phosphatase or SH2 domain with a Kd of 10⁻⁵M or less.

6. The peptide of claim 1, polypeptide of claim 2 or peptidomimetic of claim 3, wherein the phosphopeptide sequence inhibits a kinase activity with a Ki of 10⁻⁵M or less.

7. The peptide of claim 1, polypeptide of claim 2 or peptidomimetic of claim 3, wherein the phosphopeptide sequence inhibits a phosphatase activity with a Ki of 10⁻⁵M or less.

8. The polypeptide of claim 2, having an intrinsic biological activity which is regulated by the phosphorylation state of the phosphopeptide sequence(s).

9. The polypeptide of claim 2, wherein the cellular localization of the polypeptide is regulated by the phosphorylation state of the phosphopeptide sequence(s).

10. The peptide of claim 1, polypeptide of claim 2 or peptidomimetic of claim 3, covalently or non-covalently coupled to a cytotoxic agent or antiproliferative agent.

11. The peptide, polypeptide or the peptidomimetic of claim 10, wherein the agent is selected from the group consisting of alkylating agents, enzyme inhibitors, proliferation inhibitors, lytic agents, DNA or RNA synthesis inhibitors, membrane permeability modifiers, DNA intercalators, metabolites, dichloroethylsulfide derivatives, protein production inhibitors, ribosome inhibitors, inducers of apoptosis, and neurotoxins.

12. The peptide of claim 1, polypeptide of claim 2 or peptidomimetic of claim 3, coupled with an agent selected from metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

13. The peptide, polypeptide or the peptidomimetic of claim 12, coupled to a metal chelating ligand.

14. The peptide, polypeptide or the peptidomimetic of claim 13, wherein the metal chelating ligand is an NₓS_{y} chelate moiety.

15. The peptide, polypeptide or the peptidomimetic of claim 13, wherein the metal chelating ligand chelates a radiometal or paramagnetic ion.

16. An imaging preparation comprising the peptide, polypeptide or the peptidomimetic of claim 13, including a chelated metal selected from ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷ Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹²¹Sr, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu.

17. The peptide of claim 1, polypeptide of claim 2 or peptidomimetic of claim 3, coupled to a polymer or a functionialized polymer.

18. The peptide of claim 1, polypeptide of claim 2 or peptidomimetic of claim 3, formulated in a pharmaceutically acceptable excipient.

19. A nucleic acid encoding the peptide of claim 1 or polypeptide of claim 2.

20. An isolated antibody, or fragment thereof, specifically immunoreactive with a phosphopeptide sequences shown in Table 1, or corresponding phosphopeptide sequence(s) of a homologous mammalian protein.

21. The antibody of claim 20, wherein the antibody is a monoclonal antibody.

22. The antibody of claim 20, wherein the antibody is a recombinant antibody.

23. The antibody of claim 22, wherein the antibody is a single chain antibody.

24. The antibody of claim 20, wherein the antibody is labeled with a detectable label.

25. Purified preparation of polyclonal antibodies, or fragment thereof, specifically immunoreactive with a phosphopeptide sequences shown in Table 1, or corresponding phosphopeptide sequence(s) of a homologous mammalian protein.

26. A kit for detecting a phosphorylated protein comprising (i) an antibody of any of claims 20-25, or fragment thereof, specifically immunoreactive with a phosphorylated form of a phosphopeptide sequences shown in Table 1, or corresponding phosphopeptide sequence(s) of a homologous mammalian protein.

27. The kit of claim 26, wherein means for detecting the antibody is a detectable label conjugated with the antibody.

28. The kit of claim 26, wherein means for detecting the antibody is a second antibody immunoreactive with the antibody.

29. A method for identifying a treatment that modulates a phosphorylation of one or more target proteins, comprising:
(i) providing a sample including one or more peptides or polypeptides of claim 1 or claim 2;
(ii) determining the identity of peptides or polypeptides in the sample which are differentially phosphorylated in a treated sample relative to an untreated sample or control sample;
(ii) determining whether the treatment results in a pattern of changes in phosphorylation, relative to the untreated sample or control sample, which meet a preselected criteria.

30. The method of claim 29, wherein the treatment is effected by a compound.

31. The method of claim 30, wherein the compound is a growth factor, a cytokine, a hormone, or a small chemical molecule.

32. The method of claim 29, wherein the compound is from a chemical library.

33. The method of claim 29, wherein the sample is a lysates or reconsistuted protein mixture.

34. The method of claim 29, wherein the sample is a whole cell or tissue.

35. A method of conducting a drug discovery business, comprising:
(i) by the method of claim 29, determining the identity of a compound that produces a pattern of changes in phosphorylation, relative to the untreated sample or control sample, which meet a preselected criteria;
(ii) conducting therapeutic profiling of the compound identified in step (i), or further analogs thereof, for efficacy and toxicity in animals; and,
(iii) formulating a pharmaceutical preparation including one or more compounds identified in step (ii) as having an acceptable therapeutic profile.

36. The method of claim 35, including an additional step of establishing a distribution system for distributing the pharmaceutical preparation for sale, and may optionally include establishing a sales group for marketing the pharmaceutical preparation.

37. A method of conducting a drug discovery business, comprising:
(i) by the method of claim 29, determining the identity of a compound that produces a pattern of changes in phosphorylation, relative to the untreated sample or control sample, which meet a preselected criteria;
(ii) licensing, to a third party, the rights for further drug development of compounds that alter the level of modification of the target polypeptide.

38. A method of conducting a drug discovery business, comprising:
(i) providing a kinase or phosphatase assay including a peptide of claim 1 or polypeptide of claim 2, and one or more enzymes which catalyze the phosphorylation or dephosphorylation of the peptide or polypeptide;
(ii) conducting a drug screening assays to identify compounds which inhibit or potentiate the phosphorylation or dephosphorylation of the peptide or polypeptide.

39. A method of conducting a drug discovery business, comprising:
(i) providing an polypeptide including an SH2 domain which binds to a phosphorylated form of a peptide of claim 1 or polypeptide of claim 2;
(ii) conducting a drug screening assays to identify compounds which inhibit binding of the phosphorylated peptide or polypeptide with the SH2 domain.
